(19) United States
(12) Patent Application Publication
(10) Pub. No.: US 2019/0212442 A1
(43) Pub. Date: Jul. 11, 2019

(54) METHOD AND SYSTEM FOR MULTI PROBE REAL-TIME SCANNING

(71) Applicant: UC-CARE LTD., Yokneam (IL)

(72) Inventors: Tomer SCHATZBERGER, Haifa (IL); Keren SCHWEITZER, Tal-El (IL); Yeshayahu SCHATZBERGER, Haifa (IL)

(21) Appl. No.: 16/334,296

(22) PCT Filed: Sep. 18, 2017
(86) PCT No.: PCT/IB2017/055636

§ 371 Date: Mar. 18, 2019

Related U.S. Application Data
(60) Provisional application No. 62/396,829, filed on Sep. 20, 2016.

(51) Int. Cl. G01S 15/89; A61B 8/08; A61B 8/12; A61B 8/00

(52) U.S. Cl. CPC G01S 15/8936; A61B 8/08; A61B 8/12; A61B 8/4254; A61B 8/483; A61B 8/5253; G01S 15/8993; G01S 15/8995; A61B 8/4477

(57) ABSTRACT

A system and method for scanning a body anatomy. The system or method includes at least one first ultrasound transducer for collecting a first data set of the scanned anatomy, and at least one second ultrasound transducer for collecting a second data set of the scanned anatomy. Also are provided means for transforming at least one of the first and second data sets so that both scanned data sets are aligned together in a common coordinate system, preferably during a given scanning session.

### FIELD OF THE INVENTION

The invention, in some embodiments, relates to the field of treatment given to a body and more particularly, but not exclusively, to such a treatment, aided by an imaging modality that in some embodiments facilitates the registration of imaging data from several probes located in different positions in reference to the body (e.g. trans-rectal probe, abdominal probe etc.)

### BACKGROUND OF THE INVENTION

Medical procedures are commonly assisted by an imaging modality used for orientation, diagnostics and monitoring of a treatment process.

In recent years, in addition to the imaging modality, navigation systems are being employed to track a selected object, (e.g. the imaging probe) and register each image to a set of coordinates, thus enabling a 3D reconstruction and tracking of the imaging target. All the tracking methods depend within other inputs on the data acquired from the imaging modality. Lack or disrupted information as a result of the procedure—treatment physics, needle artifacts local deformations etc. reduces the tracking and monitoring ability.

For instance, US imaging is used in cryosurgical ablation, in which the advance of the ice ball can be monitored though the B-mode image, but not without limitations; The US waves are transmitted from a defined angle determined by US probe structure and the probe contact position with the body. Once the ablation begins the ice ball obstructs the US waves and therefore no imaging data can be collected beyond the ice ball front. The physician is left with a limited ability to monitor the treatment and avoid the damage for essential organs or regions. In Urological procedures for instance, limited monitoring can easily lead to damage to the Urethra sphincter or nerve bundles causing lifetime side effects to the patient.

### SUMMARY OF THE INVENTION

Aspects of the invention, in some embodiments thereof, relate to localization or mapping of a treatment given to a body. More specifically, aspects of the invention, in some embodiments thereof, relate to such a treatment, aided by an imaging modality.

In recent years there is continuous trend towards more localized treatment. A localized diagnosis enables a localized intervention, leading to reducing collateral damage during and after treatment, decreasing patient suffering and inconvenience, reducing healing time and increasing healing likelihood and reducing overall treatment cost. Local treatment procedures rely on the ability to precisely monitor the treatment process and, control the size of the treatment area to enable minimum risk for essential organs in the surroundings. The disclosed method, according to some embodiments thereof, allows for accurate location and monitoring of a region of interest in a body (e.g. region under treatment). According to some embodiments, the method allows for location and monitoring of a region of interest in a body that is more accurate than location provided by methods of prior art.

According to an aspect of the invention there is provided a method for using multi-probes for navigation and monitoring, comprising:
Providing an imaging modality comprising one or more imaging systems with at least two imaging probes designed to collect data from different positions in reference to the organ imaged. wherein the probes are configured for collecting image data of physical objects, and the image data represents a region in space corresponding to the location of the probe at the time the image data is collected;
Providing a tracking modality configured for providing data on the location of an object along pre-selected coordinates as a function of time;
Configuring the tracking modality to provide data on the location of at least one probe of the imaging modality as a function of time;
using the imaging modality and the probe thereof, collecting a first set of image data of a region in a body;
using the imaging modality and the probe thereof and at least one more probe in a different position in reference to the region in the body, collecting a second set of image data during the procedure process (e.g. biopsy, ablation treatment etc.);
Providing a registration modality, to register the second set of image data to the first set of image data to provide a complete field of view of the organ.
Providing a modality that performs local deformations correction based on the registration of the second set of image data to the first set of image data.
Providing a modality that performs local movements correction based on the registration of the second set of image data to the first set of image data.
Providing a modality that enables monitoring of the treated organ based on the registration of the second set of image data to the first set of image data.
Providing a modality that produces warnings when treatment region is in proximity to sensitive organs or regions based on the monitoring modality.
Further aspects of the present invention are exemplified in the following:
1. A system comprising of at least two imaging modality entities with at least one of them being a tracked entity; displaying an image from the combined entities.
2. A method to register image data from at least two imaging probes in different positions in reference to the imaged body/organ in real-time.
3. A method to perform deformation corrections by registration of images from at least two imaging probes in real-time.
4. A method to perform movement corrections by registration of images from at least two imaging probes in real-time.
5. A method that enables monitoring of the treated organ based on the registration of image data collected from at least two positions in reference to the imaged body/organ in real-time.

Unless otherwise defined, all technical and scientific terms used herein have the same meaning as commonly understood by one of ordinary skill in the art to which this invention pertains. In case of conflict, the patent specification, including definitions, takes precedence.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

As used herein, the indefinite articles “a” and “an” mean “at least one” or “one or more” unless the context clearly dictates otherwise.

Embodiments of methods and/or systems of the invention may involve performing or completing selected tasks manually, automatically, or a combination thereof. Some embodiments of the invention are implemented with the use of components that comprise hardware, software, firmware or combinations thereof. In some embodiments, some components are general-purpose components such as general purpose computers or oscilloscopes. In some embodiments, some components are dedicated or custom components such as circuits, integrated circuits or software.

For example, in some embodiments, part of an embodiment is implemented as a plurality of software instructions executed by a data processor, for example which is part of a general-purpose or custom computer. In some embodiments, the data processor or computer comprises volatile memory for storing instructions and/or data and/or a non-volatile storage, for example, a magnetic hard-disk and/or removable media, for storing instructions and/or data. In some embodiments, implementation includes a network connection. In some embodiments, implementation includes a user interface, generally comprising one or more of input devices (e.g. allowing input of commands and/or parameters) and output devices (e.g. allowing reporting parameters of operation and results).

### BRIEF DESCRIPTION OF THE FIGURES

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 schematically depicts a flow chart of a method for multi probe navigation, in accordance with some embodiments of the disclosure;

FIG. 2 schematically depicts an embodiment of a system configured to carry out the method of FIG. 1; and

FIG. 3A-3C schematically depict a visual representation of the multi probe registration.

### DESCRIPTION OF SOME EMBODIMENTS OF THE INVENTION

The principles, uses, and implementations of the teachings herein may be better understood with reference to the accompanying description and figures. Upon perusal of the description and figures present herein, one skilled in the art is able to implement the invention without undue effort or experimentation.

Before explaining at least one embodiment in detail, it is to be understood that the invention is not necessarily limited in its application to the details of construction and the arrangement of the components and/or methods set forth herein. The invention is capable of other embodiments or of being practiced or carried out in various ways. The phraseology and terminology employed herein are for descriptive purpose and should not be regarded as limiting.

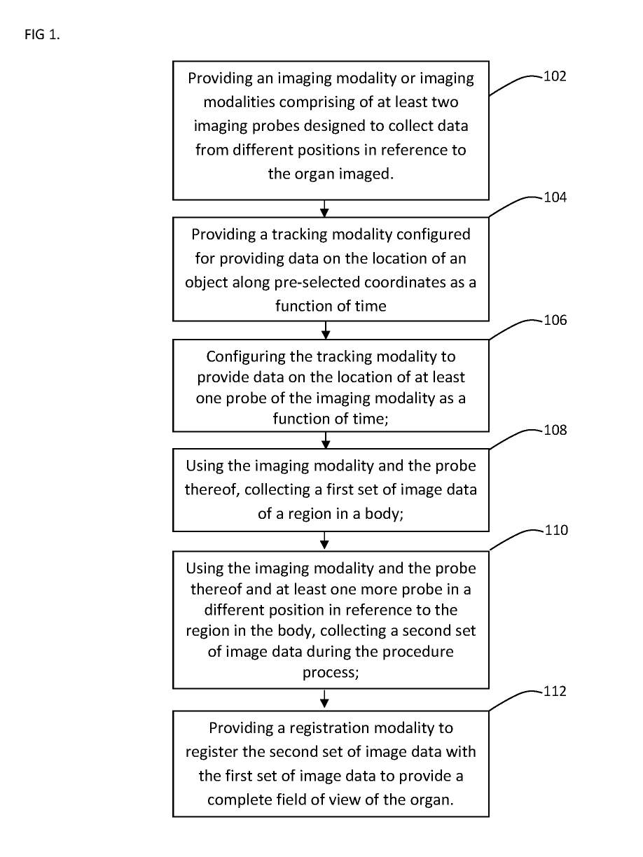

FIG. 1 schematically illustrates a flow chart of a method according to an aspect of the invention.

The method may comprise step 102 of providing an imaging modality comprising a probe (or more than one probe), wherein the probe(s) is(are) configured for collecting image data of physical objects, and the(each) image data represents a region in space corresponding to the location of the probe at the time the image data is collected.

The scanned physical object, typically a body organ, may be scanned in real-time by e.g. one, two or more probes. Such real-time scanning may be defined in at least certain embodiments as occurring substantially at the same general period of time e.g. during the same scanning session of the patient.

For example, while a patient undergoes a scanning procedure, a first probe may perform a first scan (including e.g. one or more scans) and the second probe may perform a second scan (including e.g. one or more scans) during a time period at least partially overlapping the time period of the first scan. In a further example, immediately or soon after performing the first scan, the second scan may be executed. In yet a further example, scanning sessions may be performed by a first probe, then by a second probe and then again by the first probe and/or by another probe (and so on).

Such scanning (e.g. by several probes) in real-time may provide a fuller and more complete view and understanding of internal organ anatomy, and possibly also of a real-time location and/or orientation of an invasive device inserted into the organ relative to the scanned anatomy of organ (i.e. while the scanning session is being performed).

The method may further comprise step 104 of providing a tracking modality configured for providing data on the location of an object along pre-selected coordinates as a function of time.

The method may further comprise step 106 of configuring the tracking modality to provide data on the location of the probe of the imaging modality as a function of time.

The method may further comprise step 108 of collecting a first set of image data of a first region in a body, using the imaging modality and the probe thereof.

The method may further comprise step 110 of using the imaging modality and the probe thereof and at least one more probe in a different position in reference to the region in the body, collecting a second set of image data during the procedure process;

The method may further comprise step 112 of registering the second set of image data with the first set of image data to provide a more complete field of view of the organ.

The method may further comprise step 114 of assigning a location data along the pre-selected coordinates to the second set of image data, using the correspondence of image data to the location of the probe at the time the image data is collected.

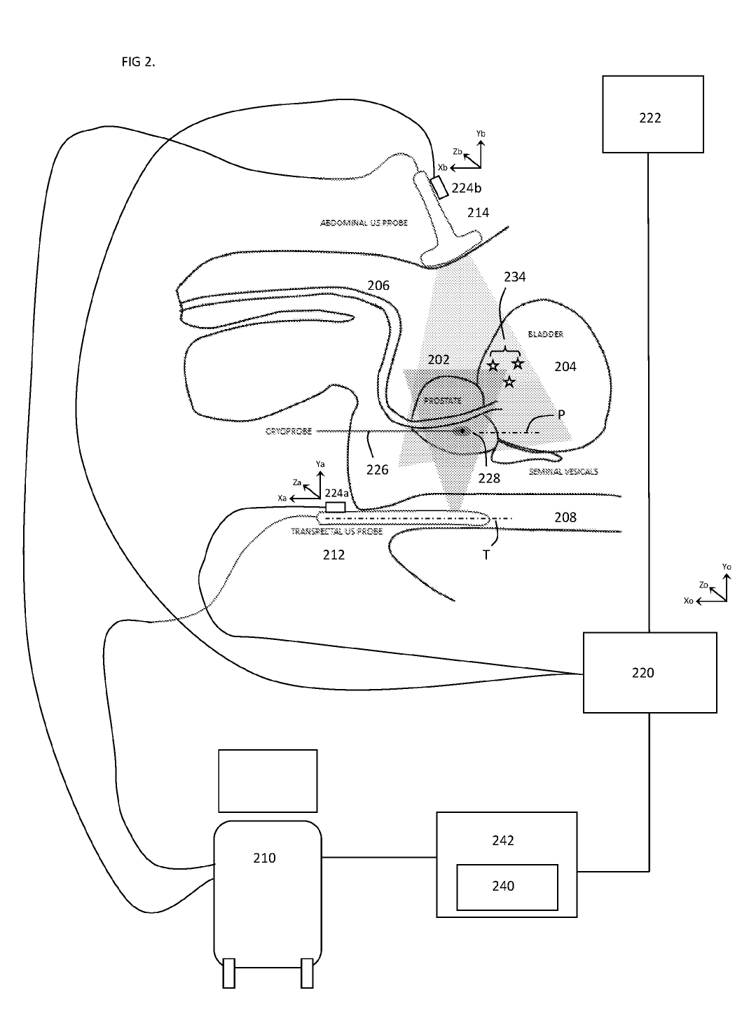

Possible anatomy, here genitourinary anatomy, of a male's body under treatment is sketched on the upper side of FIG. 2, showing the prostate 202, bladder 204, urethra 206, and rectum 208. An ultrasound scanner 210 may be in use in this example with a trans-rectal ultrasound (TRUS) transducer 212 placed in the rectum. Ultrasound scanner 210 may be configured to provide an ultrasound image obtained from ultrasound image data collected by TRUS transducer 212. For example, scanner 210 may be configured to provide an ultrasound B-mode image, which displays the acoustic impedance of a two-dimensional cross-section of tissue.

TRUS transducer 212 may be defined as having an imaginary axis T possibly along a longitudinal extension thereof and/or axis T may define a general direction along which the transducer may be axially advanced into an anatomy, here the rectum 208. Transducer 212, while being advanced along an axis generally similar to axis T and/or while being manipulated for viewing about such axis T; may view and permit grabbing of different cross sectional views of the anatomy.

In an example, TRUS transducer 212 may be positioned at a series of sequential positions along and/or about an axis generally similar to axis T in rectum 208, and collect a series of two-dimensional (2D) images.

Such images obtained by TRUS transducer 212, for example when viewing the prostate, may be utilized to obtain 3D data of the scanned anatomy, here prostate. In one example, this may be facilitated via tracking of sensor 224a by a tracking system 220. Tracking system 220, by permitting association of a local coordinate system Xa, Ya, Za (which may be fixed to sensor 224a and/or transducer 212) to each obtained 2D image; may consequently permit transformation of all the 2D images into a common coordinate system. With all the 2D images in the same coordinate system, the images may be viewed or used to provide a representation of the scanned anatomy. In addition or alternatively, the transformed 2D images may be used to create a 3D representation of the scanned anatomy.

For example, in case the obtained 2D images contain the prostate transverse sections, the images may be segmented and arranged together to obtain a 3D surface and/or 3D data set of the prostate by assigned locations provided from the tracking system 220.

An electromagnetic tracking system 220 may be configured to obtain, substantially in real time, the spatial location of suitable sensors relative to the origin of a pre-selected coordinates system Xo, Yo, Zo. In one example, coordinates system Xo, Yo, Zo may be fixed to the body of the patient so that movements of the patient may be compensated by transforming, preferably in real-time, scanned data into this coordinate system. Alternatively, coordinates system Xo, Yo, Zo may be stationary relative to the patient.

Tracking system 220 may include a transmitter 222 that produces a local electromagnetic field. Tracking system 220 may further include one or more sensors 224 such as sensor 224a, sensor 224b. Each sensor 224 may be configured to sense the EM field generated by transmitter 222 at the location of the sensor, and to obtain a signal corresponding to the sensed EM field. Upon receiving such signals from each sensor, tracking system 220 may calculate the spatial location and angular orientation of the sensor, relative to the location of transmitter 222 and/or any other point of reference, such as coordinate system Xo, Yo, Zo.

Sensor 224a may be firmly attached to TRUS transducer 212 and hence enable tracking system 220 to obtain the spatial location of TRUS transducer 212, possibly along the selected coordinates Xo, Yo, Zo that may be attached to the body. Consequently, image data collected by TRUS transducer 212, having a known spatial relation with TRUS transducer 212, may be assigned location data, as is further detailed below.

Likewise, Sensor 224b may be firmly attached (in this example) to an abdominal US transducer 214 and enable tracking system 220 to obtain the spatial location of the transducer 214 possibly along the selected coordinates Xo, Yo, Zo that may be attached to the body. Ultrasound scanner 210 in this example may be configured to provide an ultrasound image obtained from ultrasound image data collected also by abdominal US transducer 214. For example, scanner 210 may be configured to provide an ultrasound B-mode image, which displays the acoustic impedance of a two-dimensional cross-section of tissue. It is noted that possibly more than one ultrasound scanner may in some cases be used in various examples of the present disclosure, for example one ultrasound with TRUS transducer 212 and another with abdominal US transducer 214.

Tracking system 220 by permitting association (preferably in real-time) of a local coordinate system Xb, Yb, Zb to each obtained 2D image by transducer 214, may consequently permit transformation of all the 2D images into a common coordinate system. With all the 2D images in the same coordinate system, the images may be viewed or used to provide a representation of the scanned anatomy. In addition or alternatively, the transformed 2D images may be used to create a 3D representation of the scanned anatomy. Tracking system 220 may be configured to obtain, substantially in real time, the spatial location of suitable sensor 224b relative to the origin of a pre-selected coordinates system Xo, Yo, Zo.

A main controller 240 may be configured to receive ultrasound images from ultrasound scanner 210, possibly using an image grabber 242, and to receive location and orientation data of sensors 224 from tracking system 220. By using the correspondence of the image data to the location of, in this example, TRUS transducer 212 and Abdominal transducer 214 at the time the image data was collected; main controller may further be configured to assign location data to the received ultrasound images, so that substantially each pixel and/or area in an ultrasound image obtained from the image data, may be assigned a location in a coordinate system attached to the body under treatment, such as coordinate system Xo, Yo, Zo.

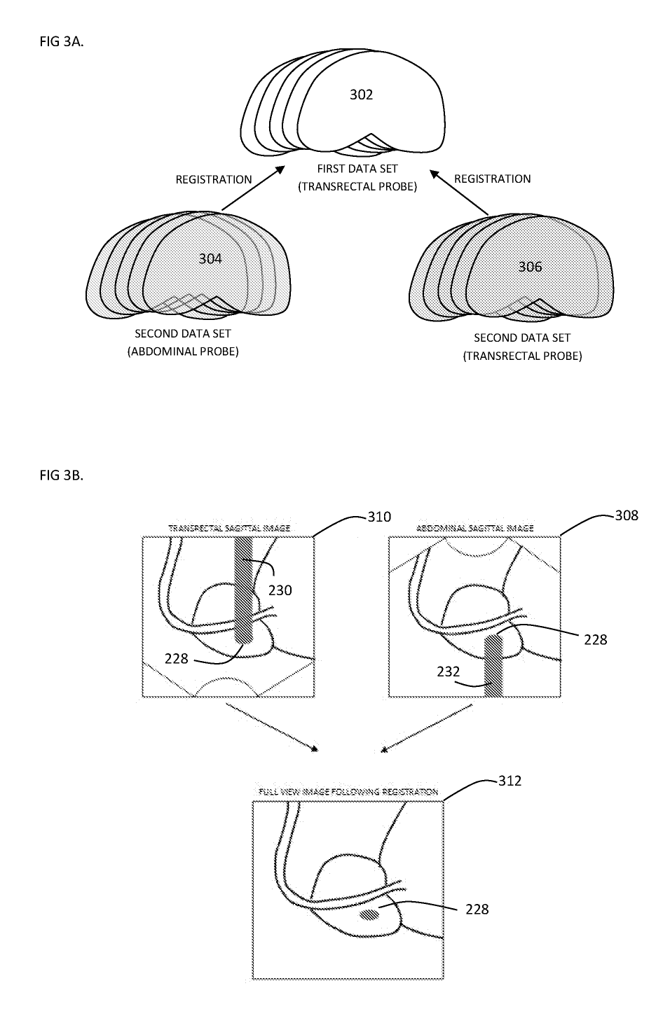

By having within the same scanning session the real-time location data for several transducers, here both transducers (212, 214), assigned to each one of the US images; the images may be segmented and arranged together to obtain two 3D surfaces and/or 2D image data sets (304, 306) of the scanned anatomy, here prostate, from two different view angels. For example, in a focal Cryo-ablation treatment scenario, an initial scan from e.g. transducer 212 can be performed pre-treatment and a 3D model may be obtained as described above (302). During treatment, two or more additional scans may be performed to monitor the treatment, the additional scans may be performed with transducers 212 and 214, and 3D surfaces and/or 2D image data sets may be obtained (304,306). The main controller may register these surfaces and/or image data to the initial scanned data, e.g. 3D surface obtained pre-treatment. Registration of the data from e.g. the two transducers may enable to complete any missing data due to the obstruction of the US waves by the treatment (e.g. ice ball in Cryo-ablation) [FIG. 3b].

With attention drawn back to FIG. 2, an invasive device such as a needle-like applicator 226 (i.e. a cryoprobe) is illustrated in place in the prostate removing heat from its tip and by extension from the surrounding tissues causing an ice ball Cryo-ablation obstruction 228. In FIG. 2, transducers 212 and 214 are illustrated scanning the area of the ablation from different angles.

With attention additionally drawn to FIG. 3B, respective two images obtained by transducers 212 and 214 are illustrated at the top of this figure. The upper left image schematically illustrates a ‘shadow’ 230 created generally above and behind the obstruction 228 when viewed generally from below by transducer 212. The upper right image schematically illustrates a ‘shadow’ 232 created generally below and behind the obstruction 228 when viewed generally from above by transducer 214.

The ‘shadows’ 230, 232 represent areas/data that may generally be lacking in both images due to obstruction 228. Thus, by transforming data from these different views into a common coordinate system, possibly as discussed above or below, the scanned information from the different angles may be aligned to provide a ‘fuller’ and more complete view of the scanned anatomy e.g. by using scanned data from one view to ‘fill in’ data that was missing in the ‘shadowed’ area in the other view. Such a ‘fuller’ view is schematically illustrated at the bottom image in FIG. 3B.

Although a cryoprobe applicator has been discussed, other types of procedures causing such ‘shadows’ may also benefit from the herein discussed real-time alignment methods. For example, application of heat in order to treat tissue in a scanned anatomy may also cause such ‘shadows’ that may then be filled in as discussed.

It is noted that other means/methods for aligning data scanned in real-time by transducers, such as 212, 214, into a common coordinate system; may be used in addition or in alternative to the above discussed method utilizing the tracking system 220. For example, scanned data by each transducer may be used to create a 3D local data set and then alignment between the two (or more) 3D data sets may be performed by e.g. best fitting the 3D data set(s) one to the other. In a further example, implanted land marks 234 (e.g. fiducial markers) may be placed in the scanned anatomy to be in a field of view of the transducers (e.g. 212, 214) and consequently in the images produced by the transducers, for use as points of reference for alignment of scanned 2D and/or 3D sets one to the other. Alignment between scanned 2D and/or 3D sets may also be performed on basis of common anatomy identified in both data sets and consequently used for defining the alignment.

It is noted in addition that while the transducers 212,214 are illustrated and explained herein above as abdominal and rectal, other types of transducers may equally be used that are sized and shaped for scanning other parts of the body. For example, at least one of the transducers may be configured for scanning anatomy, such as genitourinary anatomy, from a direction of the perineum.

It is yet further noted that scans made by the transducers in real-time within the same scanning session, may not necessarily be obtained at the same point in time. For example, a 2D and/or 3D data set obtained by one transducer, e.g. 212, may be initially performed; and thereafter an additional 2D and/or 3D data set obtained by another transducer, e.g. 214 (or even the same transducer e.g. from another direction), may be performed; and possibly aligned to the previously obtained data set(s).

In therapeutic procedures involving positioning a probe (e.g. cryosurgical probe) in a patient's body, it is typically required to perform an accurate positioning of the probe within the body so that a surgeon can be provided with sufficient information for performing the procedure. For example, alignment between an ultrasonic probe and the cryosurgical probe may be performed prior to the surgical procedure so that the surgeon ensures he has visual determination that the probe is positioned directly above and in the path of the energy beam generated by the transducer so that the cryosurgical probe can be viewed during the procedure. Consequently, the longitudinal axes of the probe and transducer are typically arranged to be in spaced parallel alignment one above the other.

In an aspect of the present invention, the real-time alignment procedures of data obtained by the transducers (such as 212, 214) in the same scanning and/or therapeutic session; may permit performing a surgical procedure where the axes of the probe and transducer monitoring the probe, are not necessarily arranged to be in spaced parallel alignment one above the other—while still providing the surgeon with sufficient information for performing the procedure by superimposing in real-time data sets obtained from different views one on top of the other. In the example shown in FIG. 2, this may be seen e.g. in axis T of transducer 212 and an axis P of applicator 226 not necessarily being arranged to be in spaced parallel alignment one above the other.

In one example, this may be facilitated by 2D and/or 3D data obtained by the transducers being brought, via the discussed real-time alignment procedures, into a common coordinate system where all obtained information can be viewed in the same reference system. Thus, e.g., discrete segments of applicator 226 appearing in different cross sectional 2D images (if such alignment is lacking between axes P, T); may be gathered together during a scanning session to form a more complete view of the applicator when the data of the images is aligned into the common coordinate system. A surgeon, viewing e.g. in a 3D model the applicator and treated anatomy may then be able to determine e.g. the location of the applicator's tip in relation to the anatomy in order to execute the therapeutic procedure.

1. A system for scanning a body anatomy, the system comprising:
at least one first ultrasound transducer for collecting a first data set of the scanned anatomy,
at least one second ultrasound transducer for collecting a second data set of the scanned anatomy, and
means for transforming at least one of the first and second data sets so that both scanned data sets are aligned together in a common coordinate system, preferably during a given scanning session.

2. The system of claim 1 and comprising at least one ultrasound scanner for providing ultrasound images obtained from the first and second ultrasound transducers.

3. The system of claim 2 and comprising a tracking system for tracking each one of the ultrasound transducers for assisting in the alignment.

4. The system of claim 3 wherein the common coordinate system is attached to be movable with the scanned body.

5. The system of claim 4 wherein each one of the first and second data sets is a 2D data and/or a 3D data set.

6. The system of claim 5 wherein each scanned data in the first and second data sets is associated with its relative spatial location within the common coordinate system and/or a local coordinate system later transformed into the common coordinate system.

7. The system of claim 6 wherein association of the relative spatial location is performed in real time while obtaining the data by the transducers.

8. The system of claim 7 and comprising at least one sensor attached to each transducer for being tracked by the tracking system in order to obtain the spatial location of the transducer in real time and hence of data obtained by the transducer in real time.

9. The system of claim 8 and comprising a controller configured to receive 2D data from the ultrasound scanner and location and orientation data of sensors attached to the transducers from the tracking system.

10. The system of claim 9, wherein the controller is further configured to correspond 2D data to the location of the transducer that grabbed the 2D data at the time the 2D data was collected.

11. The system of claim 2 wherein the alignment is performed by using common anchoring information identified within at least some of the data of the first and second data sets.

12. The system of claim 11 wherein the anchoring information is at least one of: implanted land marks and/or common anatomy identified in both data sets.

13. The system of claim 1, wherein the first ultrasound transducer is an abdominal transducer and the second ultrasound transducer is a trans-rectal ultrasound transducer.

14. A method for scanning a body anatomy comprising the steps of:
providing a system comprising at least two ultrasound transducers,
collecting in real-time data sets of the scanned anatomy by the transducers, and
transforming in real-time data collected by at least one of the transducers so that data sets obtained by both transducers are aligned together in a common coordinate system.

15. The method of claim 14, wherein the real-time alignment of data to the common coordinate system is during the scanning of the body anatomy, preferably within a single scanning session.

16. method of claim 11 or 15, wherein the system further comprising a tracking system for tracking each one of the ultrasound transducers for assisting in the alignment.

17. The method of claim 16, wherein data collected by a given transducer is first associated with local coordinate system fixed to the given transducer and possibly later transformed to the common coordinate system, wherein preferably the association of the local coordinate system to the collected data is in real-time during the scanning session.

18. The method of claim 15, wherein real-time collecting of data sets of the scanned anatomy by the transducers is by a first one of the transducers performing a first scan and a second one of the transducers performing a second scan during a time period that: at least partially overlaps the time period of the first scan and/or occurs soon after performing the first scan.

19. A method for treating an anatomy of a body comprising the steps of:
scanning the body anatomy by at least two ultrasound transducers within the same given scanning session, and
transforming data collected by at least one of the transducers so that data sets obtained by both transducers are aligned together in a common coordinate system.

20. The method of claim 19 and comprising a step of performing an invasive procedure of entering an invasive device into the anatomy of the body, wherein the invasive device is scanned by both transducers to receive in real-time, preferably during the execution of the procedure, a more complete view of the device in scanned data aligned into the common coordinate system.

21. The method of claim 20, wherein the invasive device has a longitudinal extension extending along an axis P and at least one of the transducers has a longitudinal extension extending along an axis T; and at least a portion, preferably substantially all, of the invasive device along its longitudinal extension is identified in aligned scanned data in the common coordinate system while the axes P and T are not arranged to be in spaced parallel alignment one above the other.

22. The method of claim 21, wherein the invasive device is a needle-like applicator such as a cryoprobe and the transducers extending along an axis T is a trans-rectal ultrasound transducer placed in the rectum.
